(19) United States
(12) Patent Application Publication
(10) Pub. No.: US 2020/0039178 A1
(43) Pub. Date: Feb. 6, 2020

(54) STRETCHABLE COMPOSITE SHEET, METHOD FOR MANUFACTURING A STRETCHABLE COMPOSITE SHEET, AND APPARATUS FOR MANUFACTURING A STRETCHABLE COMPOSITE SHEET

(71) Applicant: Sun Tool Corporation, Moriguchi shi, Osaka (JP)

(72) Inventor: Shoji HIDAKA, Moriguchi shi (JP)

(73) Assignee: Sun Tool Corporation, Moriguchi shi, Osaka (JP)

(21) Appl. No.: 16/339,302

(22) PCT Filed: Oct. 2, 2017
(86) PCT No.: PCT/JP2017/037285

§ 371 Date: Apr. 3, 2019

(30) Foreign Application Priority Data
Oct. 5, 2016 (JP) 2016-209051

(51) Int. Cl. B32B 5/02; A61F 13/49; B32B 5/26

(52) U.S. Cl. CPC B32B 5/022; B32B 5/26; A61F 13/49

(57) ABSTRACT

A stretchable composite sheet in which a plurality of stretchable elastic members (rubber threads) are adhered and fixed to where peaks of pleats (folds) of two non-woven fabric sheets and the plurality of the stretchable elastic members (rubber threads) are in contact therewith, with the stretchable elastic members (rubber threads) being positioned in an orthogonal direction with respect to the peaks of the double-layer pleats (folds). Coating of adhesive is applied to the entire circumferential surfaces of the stretchable elastic members (rubber threads) during the intermittent conveyance of the stretchable elastic members. An apparatus for manufacturing the stretchable composite sheet is provided with a coater application head which applies adhesives through slit grooves thereof to each of the stretchable elastic members (rubber threads) passing through the slit grooves.

### TECHNICAL FIELD

The present invention relates to a stretchable composite sheet in which gathers and pleats that are flexible and has a good feel and pleasing to the touch can be formed.

### BACKGROUND TECHNOLOGY

For absorbent articles such as disposable diapers, feminine hygiene napkins, etc., it is widely employed to use a method in which an elastic member in a stretched state is joined to a sheet material and then gathers, pleats (which are expanding and contracting areas having a great number of folds) are formed on the sheet material by causing the elastic member to contract.

Patent Document 1 discloses, as seen from FIGS. 15, 16, a disposable diaper. This diaper includes an elastic stretchable part that is comprised of a non-woven fabric sheet material F and elastic members sandwiched between a two-layer part formed by folding the non-woven fabric sheet material. The elastic stretchable part has linear joining portions HM′ which are comprised of the sheet materials extending in a direction perpendicular to the stretching direction of the elastic members, and the elastic members R are joined to the sheet material at these linear joining portions.

### PATENT DOCUMENTS

Patent Document 1: Japanese Registered Utility Model No. 2518953

### Problems the Invention is to Solve

In this elastic stretchable part described in Patent Document 1, the sheet bulges between the linear joining portions, thus forming homogenous pleats. However, the flexibility in the direction that is parallel with respect to these linear joining portions is inferior due to these linear joining portions, and as a result there are problems in terms of softness and feel when they comes in contact with the skin.

More specifically, linear coating areas are formed by applying adhesives to sheet materials, and a plurality of elastic members (rubber threads) are adhered at their one side to the linear coating areas, thus fixing the two sheet materials together and fixing the two sheet materials and the rubber threads mutually together. Accordingly, because of the linear coating areas, problems occurs in terms of the usage of a diaper formed with pleats.

Accordingly, the object of the present invention is to eliminate the above-described problems by forming linear joining areas comprised of two sheet materials and rubber threads in a manner of making a point contact and to strengthen the mutual adherence of the two sheets and rubber threads.

### Means to Solve the Problem

The first invention of the present application relates to a stretchable composite sheet in which a plurality of stretchable elastic members (rubber threads) are adhered to and fixed between two non-woven fabric sheets, so that double-layer pleats (folds) are formed in the orthogonal direction with respect to the plurality of stretchable elastic members (rubber threads), and this stretchable composite sheet is characterized in that
the plurality of stretchable elastic members (rubber threads), positioned in the orthogonal direction with respect to the peaks of the double-layer (folds), are adhered and fixed at both sides thereof to the two non-woven fabric sheets at contact points where the plurality of stretchable elastic members (rubber threads) and the peaks of the pleats (folds) are in contact.

The second invention of the present application relates to a method of manufacturing a stretchable composite sheet, and the method is characterized in that
when an intermittent coating of an adhesive to a plurality of stretchable elastic members (rubber threads) before these stretchable elastic members (rubber threads) are fed into between two non-woven fabric sheets, the coating of the adhesive is made to the entire circumferential surface of the stretchable elastic members (rubber threads) while the stretchable elastic members are being conveyed.

The third invention of the present application relates to an apparatus for manufacturing a stretchable composite sheet, and the apparatus is characterized in that
the apparatus includes an adhesive intermittent coating device provided with a coater head that is formed with a plurality of slit grooves;
each of the slit grooves is formed at a base portion thereof with an expanded section for coating an adhesive to each of the stretchable elastic members (rubber threads) passing through the slit grooves, the expanded section being larger than a cross section of each of the stretchable elastic members (rubber threads), thus making an adhesive coating area; and
the apparatus supplies the adhesive from both sides of the adhesive coating area.

### Advantageous Effects of the Invention

In the stretchable composite sheet according to the present invention, the joining area of the rubber thread to the non-woven fabric is in a relation of a point contact, and therefore, in comparison to a linear contact, the stretchable composite sheet of the present invention is advantageous in that it has pleats (stretchable parts having a plurality of folds) which are flexible with a good feel and pleasing to the touch to a skin, making it possible to form such pleats in absorbent articles and the like, and it is also possible to reduce the volume of coating.

Furthermore, although the joining area is in a point contact, the rubber threads are adhered and fixed to two non-woven fabrics with both top and bottom surfaces thereof, and therefore, the stretchable composite sheet has such an effect that the two sheets of non-woven fabrics and the rubber threads are strongly joined.

### BRIEF DESCRIPTION OF THE DRAWINGS

FIG. 1A is a perspective view of a stretchable composite sheet according to one embodiment of the present invention, FIG. 1B is a longitudinal cross-sectional view of a part thereof at a location where a rubber thread is adhered.

FIG. 2A is a top plan view of the stretchable composite sheet in a stretched state, FIG. 2B is an enlarged longitudinal cross-sectional view of a part thereof taken along the lines S2B-S2B of FIG. 2A, and FIG. 2C is an enlarged horizontal cross-sectional view taken along the lines S2C-S2C of FIG. 2B.

FIG. 3A is a side view of a rubber thread in a stretched state, and FIG. 3B is an enlarged longitudinal cross-sectional view thereof.

FIG. 4 A schematic front view of a manufacturing line according to the present invention for manufacturing a stretchable composite sheet.

FIG. 5 A top view thereof.

FIG. 6 A longitudinal cross-sectional view of an intermittent coating device used in the manufacturing line.

FIG. 7A is a schematic perspective view of a coater head installed at a lower end of the intermittent coating device, FIG. 7B is longitudinal cross-sectional view thereof, and FIG. 7C is a front view thereof.

FIG. 8 A side view thereof.

FIG. 9 A longitudinal cross-sectional view of the coater head taken along the line S9-S9 of FIG. 8.

FIG. 10 A longitudinal cross-sectional view taken along the line S10-S10 of FIG. 8.

FIG. 11A is a top view of a stretchable composite sheet with the stretchable elastic members (rubber threads) in the stretched state, and FIG. 11B is an enlarged cross-sectional view thereof.

FIG. 12 A cross-sectional view of a cam-style suck-back coating gun unit at a time when the operation is OFF, showing an intermittent coating means of adhesives to coat adhesives to rubber threads.

FIG. 13 A cross-sectional view of the cam-style suck-back coating gun unit at a time when the operation is ON.

FIG. 14 An explanatory diagram of an intermittent coating.

FIG. 15 A perspective view of a known stretchable composite sheet in partially cross-sectioned.

FIG. 16A is a top view of a known stretchable composite sheet in a stretched state, and FIG. 16B is an enlarged cross-sectional view thereof.

### PREFERRED EMBODIMENTS OF THE INVENTION

In the present invention, stretchable elastic members (rubber threads), to which adhesives have been applied to the entire circumferential surfaces at locations where coating of adhesives is made, are supplied, so that the adhesive coated locations are supplied to contact points where the elastic members are in contact with the peaks of the pleats (folds) of two non-woven fabrics.

As a result, being set in the orthogonal direction with respect to the peaks of double-layer pleats (folds), the plurality of stretchable elastic members (rubber threads) are adhered and fixed at both sides thereof to contact points of the plurality of stretchable elastic members (rubber threads) and the pleats (folds) of two non-woven fabrics.

A detailed description will be made based on the embodiments of the present invention.

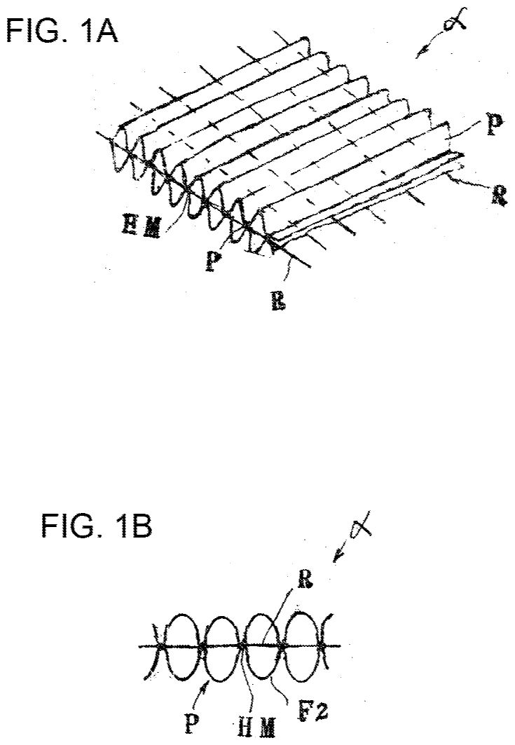

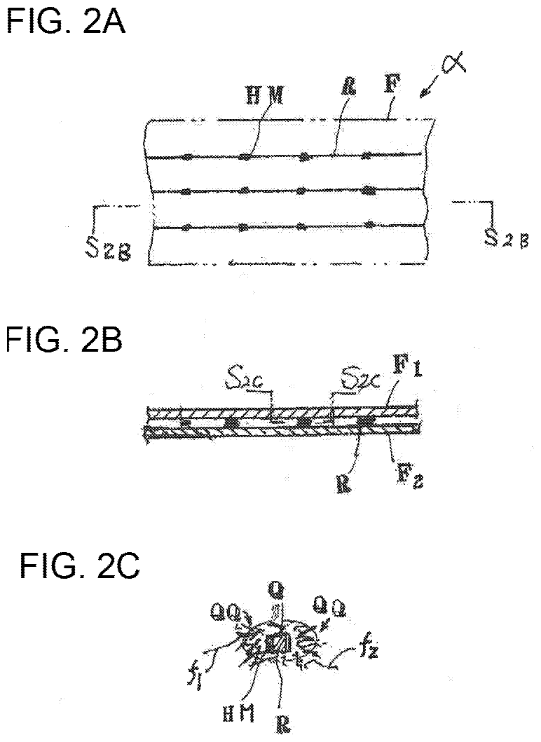

FIG. 1 is a perspective view of a stretchable composite sheet α in a state in which pleats P have been formed in an implementation of the present invention. FIG. 2 shows a stretchable composite sheet α in a state stretched, wherein FIG. 2A is a top plan view thereof, and FIG. 2B is a longitudinal cross-sectional view thereof taken in the length-wise direction.

A plurality of stretchable elastic members (rubber threads) R in its stretched state are disposed between two sheets of non-woven fabrics F at points at which hot melt adhesives HM are applied on the entire circumferential surface intermittently at specified intervals on stretchable elastic members (rubber threads) R.

With this arrangement, the plurality of stretchable elastic members (rubber threads) R are adhered and fixed at both sides thereof to the non-woven fabric F at contact points where the peaks of the pleats (folds) P and the plurality of stretchable elastic members (rubber threads) R are in contact, which are positioned in a orthogonal direction with respect to the peaks of the two-layer pleats (folds).

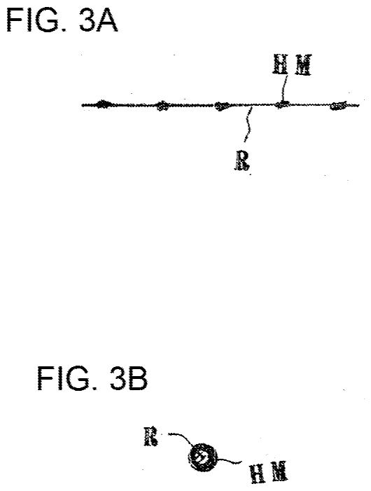

FIG. 3 shows the stretchable composite sheet α, in which the plurality of stretchable elastic members (rubber threads) are adhered and fixed to two-layer non-woven fabric sheets and then contracted to their non-stretched (natural) length, so that the plurality of stretchable elastic members (rubber threads) are adhered to and fixed between the two sheets of non-woven fabrics, and pleats (folds) in two, above and below, layers are formed in the orthogonal direction with respect to the plurality of stretchable elastic members (rubber threads).

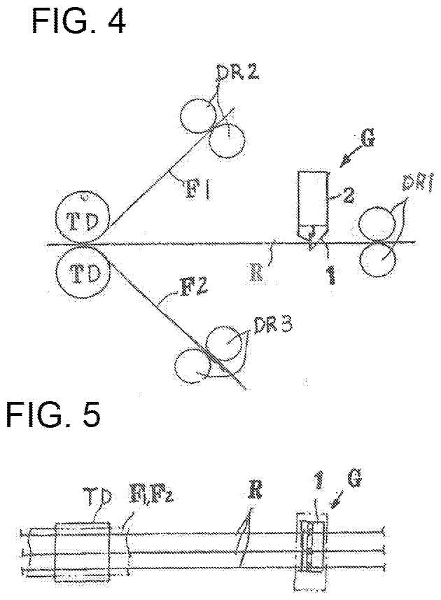

FIG. 4 shows stretchable elastic members (rubber threads) R used in the manufacturing of the stretchable composite sheet of the present invention. The stretchable elastic members (rubber threads) is shown in its stretched state and at specified hot-melt adhesive coating locations adhesives HM are applied to the entire circumferential surface of the stretchable elastic members (rubber threads).

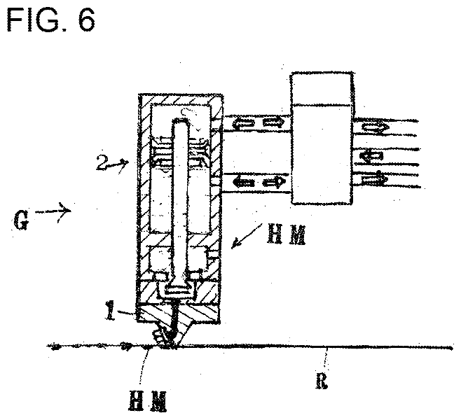

The manufacturing line of the stretchable composite sheet will be described below with reference to FIG. 5 and FIG. 6.

The manufacturing line is comprised of an adhesive intermittent application step and a stretching step that follows the adhesive intermittent application step. In the adhesive intermittent application step is provided with a coating gun unit G that is comprised of an adhesive intermittent application device 2 and a coater application head 1 which is disposed at the tip end of the application device 2.

Using the coater application head 1 of the coating gun unit G, an adhesive HM is intermittently coated to the rubber thread R which is fed in an stretched state by the drive rolls DR1, DR1.

In the stretching step, a first sheet F1 is fed by drive rolls DR2 onto the upper side of the rubber thread R on which the adhesive HM has been intermittently applied, and a second sheet F2 is fed by a drive roll DR3 onto the underside of the rubber thread R on which the adhesive HM has been intermittently applied.

The first sheet F1, the rubber thread R on which the adhesive HM has been applied, and the second sheet F2 are supplied to between a stretching roll (nip roll) DT1 and another stretching roll DT2. As a result, the first sheet F1, the second sheet F2 and the rubber thread R are unified by the adhesive HM, forming a stretchable composite sheet α, making the rubber thread R in the stretched state. In this stretchable composite sheet α with its rubber thread R in the stretched state, as seen from FIG. 3C, an adhesive area Q of the rubber thread R is surrounded by fixed areas QQ.

In this fixed areas QQ, fibers f1 of the lower layer of the first sheet F1 and fibers f2 of the upper layer of the second sheet F2 are entangled with each other with the adhesive HM adhering thereto in an integrated fashion and there exist sheet spaces h that penetrate up and down.

In the next step, the stretching power of the rubber thread R in the stretched state is released, so that the rubber thread R is brought into its normal length (natural length), thus completing the stretchable composite sheet as shown in FIG. 1, FIG. 2.

The coater application head 1 used in the above-described adhesive intermittent application step will be described below.

As seen from FIGS. 6-10, the coater application head 1 is composed of a head main unit 11, a presser plate (blind plate) 12, and a shim plate (distribution plate) 13. In the head main unit 11, an adhesive supply hole 18 and a distribution feed passage 14 are formed. A hot melt adhesive is supplied to an adhesive coating area E of a slit groove 15 of the shim plate 12, and a rubber thread(s) R and the like is supplied to pass through the adhesive coating area E of the slit groove 15 as in the conventional structurer.

In the implementation of the present invention, a communication passage 17 is, for the purpose of supplying hot-melt adhesive, is provided on each of the left and right sides of the adhesive coating area E.

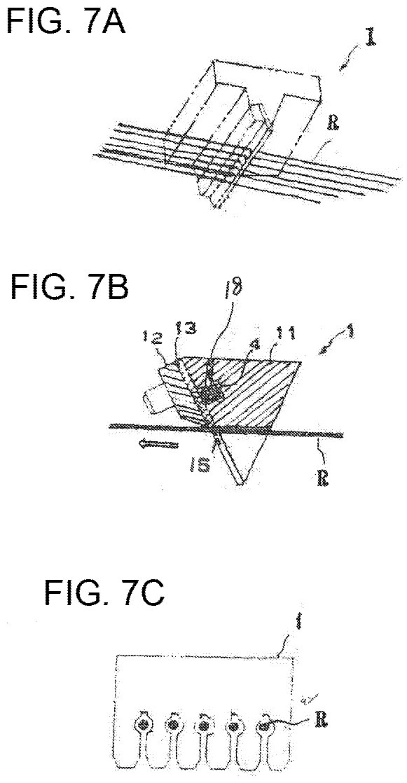

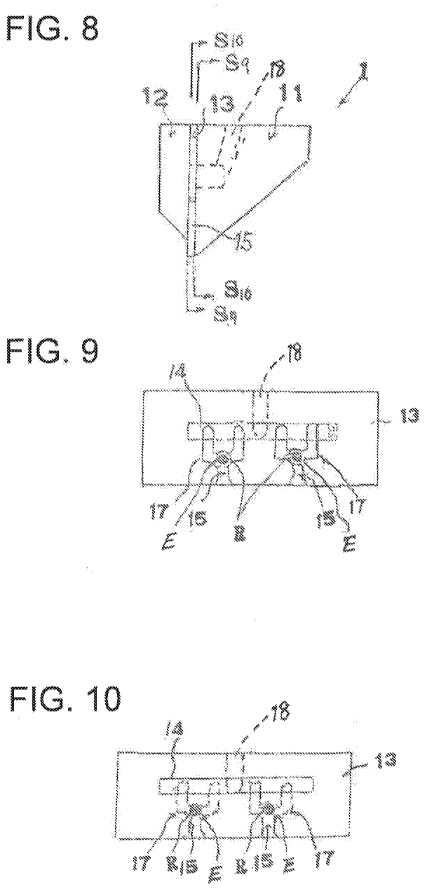

The communication passages 17 make a pair on the left and right sides of the slit groove 15. In addition, the slit groove 15 can be provided for only one, or it can be provided in a plurality of numbers such as two (2), four (4), etc. In the structure of FIG. 7A, a single slit groove 15 is provided, and in the structure of FIG. 8A, six (6) slit grooves 15 are provided.

In the present invention, a plurality of stretchable elastic members (rubber threads) are respectively adhered and fixed at both sides thereof to non-woven fabric sheets, and the fixed portions of the stretchable elastic members are provided in a dot format in a direction perpendicular to the length-wise direction of the stretchable composite sheet.

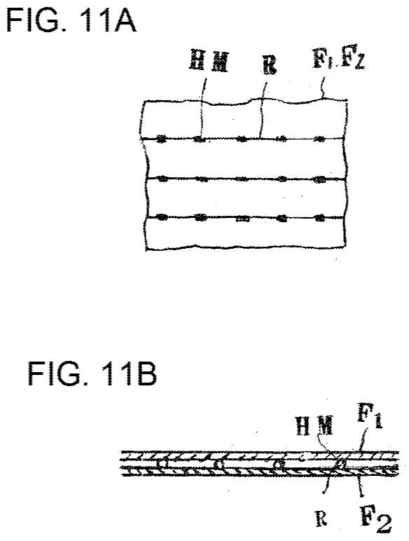

Next, with reference to FIGS. 11 to 13, an adhesive intermittent supply device (coating gun unit main body) 2 of the adhesive intermittent application gun unit G will be described.

The coating gun unit main body 2 is comprised of a valve mechanism A, which includes a movable valve element 3 therein, and a cam-type valve driving mechanism (valve element driving mechanism) B, which includes a cam body 6 that moves the moveable valve element 3 up and down; and the valve mechanism A and the valve driving mechanism B are integrated to form a single unit.

The moveable valve element 3 is supported by a bearing 32 which is provided inside the housing 10, so that the valve element 3 is movable up and down.

The housing 10 has a valve chamber 33 in the lower part thereof, and the valve chamber 33 is provided therein with a valve seat 34 on the bottom surface of the valve chamber 33. The housing 10 further includes an adhesive inlet 35 formed on its side wall so as to communicate with the inside of the valve chamber 33.

The valve mechanism A is thus structured as described above, so that it has the above-described suck-back type intermittently supply function between the valve seat 34 and an expanded portion 31 of the valve element 3.

The top end of the moveable valve element 3 is fixed to the lower part of the moving body 41 by a fastening device (nut) 45, and the lower end of a needle 5 is fixed to the upper part of the moving body 41 by a fastening device (nut) 46. The moveable valve element 3 and the needle 5 are thus integrated.

A bearing 47 is installed in the upper end of the housing 10.

The needle 5, which is fixed at its bottom end to the upper portion of the moving body 41, is provided to pass through the bearing 47, so that the needle 5 is guided thereby for its up and down movements.

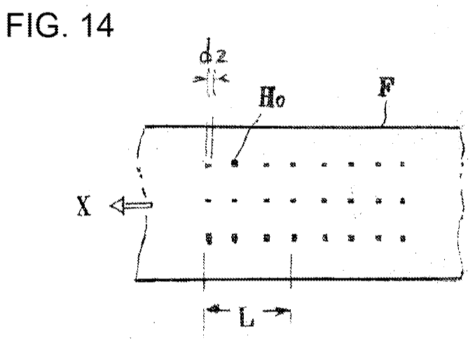

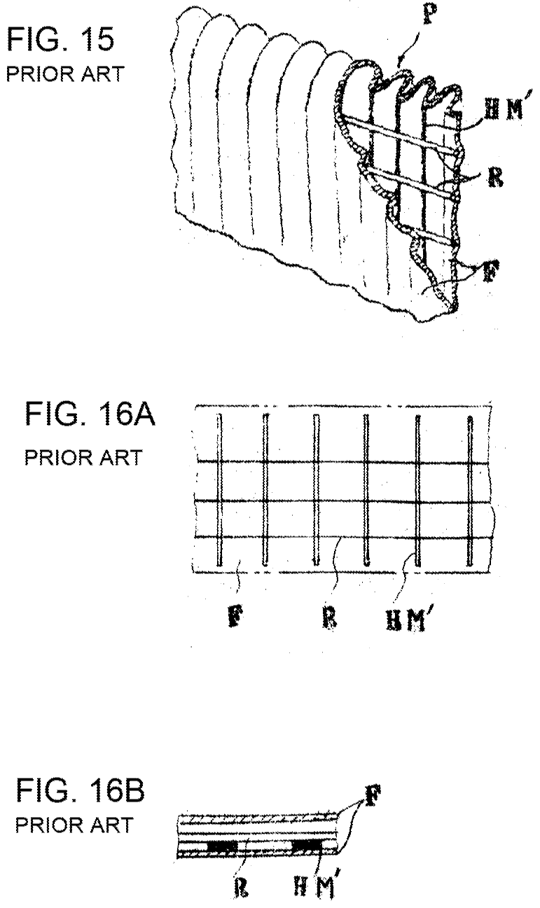

In the shown embodiment, as seen from FIG. 14, the circumferential length L of the cam surface 61 of a cam body 6 when it rotates once is set to be an up and down stroke P2 of the needle 5.

The upper end of the needle 5 is set to be in contact with the cam surface 60 of the cam body 6, so that the rotation of the cam body 6 is converted into the up and down movement of the needle 5.

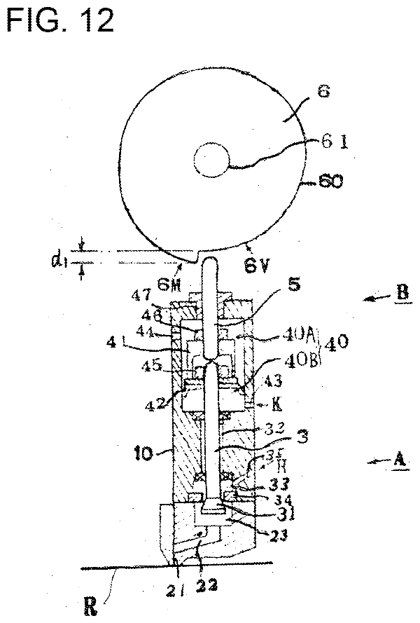

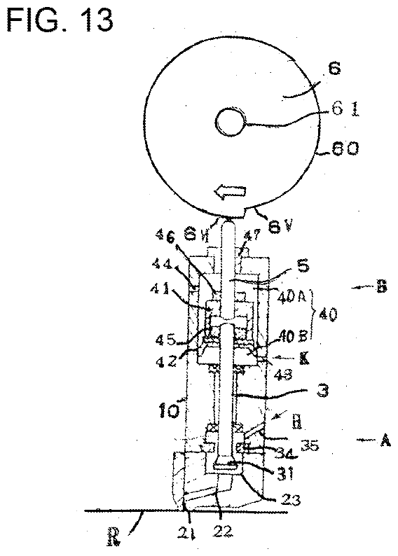

In FIGS. 12 and 13, the reference numeral 61 is a drive shaft for the cam body 6.

FIG. 14 shows a case in which the cam body 6 is comprised of equally divided four parts under the conditions noted below, so that one rotation of the cam body 6 provides four strokes.
Stroke of needle: d₁=0.4 mm
   Cam body: Divided into four parts
   Cam rotation: 400 rpm
   Servomotor ON/OFF
      operation time: 30 ms
   Response frequency: 2000/min.
   Coating length: d₂=2.5 mm
   Coating pause length: 7.0 mm
   Cam surface length by
      one rotation of cam body: L=40 mm

The characteristics of the cam-type valve driving mechanism (valve element driving mechanism) B that includes the cam body 6 are as follows:
The rotational drive of the cam body 6 is adjustable for the speed by a servomotor, and thus it is possible to electrically control the coating length.
A response speed that exceeds the limitations which an solenoid valve provides is achievable.
The rotation of the cam body 6 and the stroke action are directly linked and synchronized.
The cam body 6 is a circular disk, and the coating length is determined by peaks and valleys formed arbitrarily.
The rotational drive of the cam body 6 can be made by a servo motor with the rotational speed being variable, thus controlling the coating length electrically.
The rotation of the servomotor is coupled to a base unit, and the coating locations can be set thereby.
The rotation of the servomotor in the range of 0-3000 rpm per minute can be used for the ON-OFF operation of the cam body.
There is no operational loss time since the stroke of the needle and the movable valve element is made directly by the rotation of the cam body and not by a solenoid valve and air.
Solenoid valve connecting parts are not required.
Parts replacement work hours and parts replacement costs resulting from the response life of a solenoid valve are eliminated
By dividing the 360-degree single rotation into four parts for the cam body, it is possible to increase the ON-OFF range of the cam body.
In driving made by a solenoid valve and air, a stroke of 1400/minute has been the limit; however, in the rotational driving made by a servomotor, a stroke of 3000/minute of the needle and the movable valve element becomes possible.

When coating the adhesive to a rubber thread(s) R, etc., the adhesive is supplied to the adhesive supply hole 18 of the coater application head 1 through the valve mechanism V from a hot-melt supply source. Then, the adhesive is supplied to the expanded section E of the slit groove(s) 15 through the distribution feed passage 14 and communication passage 17 of the coater application head 1. In these processes, the adhesive is fed to the entire circumferential surface of the rubber thread(s) R with a specified coating timing by means of an intermittent ON/OFF operation of the valve mechanism V.

When the intermittent operation of ON/OFF of the valve mechanism 2 is done, the adhesive is sucked up by the suction action at the time of OFF operation due to the suck-back effect of the valve mechanism, and the timing of coating interruption is made clear.

In the shown embodiment, the valve mechanism 12 is structured so that the up and down movements of the piston 14 which is integrated with the valve member 13 selectively supply the operation air from the operation air source K in the form of an ON movement operation air K1 or an OFF movement operation air K2 through a solenoid valve 16. It is also possible to use a solenoid valve instead of the cam-driving-type valve mechanism.

In the ON operation of the valve mechanism, with the provision of the enlarged part E in the top end (base part) of the slit groove 15 and with the supply of the adhesive via the communication passages 17 provided on both left and right sides of enlarged part E, the adhesive can be, quickly upon the ON operation timing, coated on the entire circumferential surface of the elastic string-like body that is passing through the enlarged parts E of the above-described slit groove.

In addition, in the OFF operation of the valve mechanism, the adhesive in the enlarged part E is sucked in by the suck-back effect, and coating is thus interrupted. Accordingly, the setting of the coating location and coating length can be made precise.

### INDUSTRIAL APPLICABILITY

The present invention provides pleats that are flexible with a good feel and pleasing to the skin as a composite stretchable member in the manufacture of paper diapers, making it possible to form optimal pleats for absorbent articles and the like and having the effect of reducing the volume of coating of adhesives. The present invention thus contributes to the development of manufacturing industries of paper diapers, etc.

### EXPLANATION OF SYMBOLS

α Stretchable composite sheet
DR Drive roll
F Non-woven fabric
HM Hot melt adhesive
G Coating gun unit
R Stretchable elastic member (rubber thread)
P Pleats (folds)
TR Stretching roll

1. A stretchable composite sheet in which a plurality of stretchable elastic members are adhered to and fixed between two non-woven fabric sheets so that a double-layer pleats (folds) are formed in an orthogonal direction with respect to the plurality of stretchable elastic members (rubber threads), characterized in that:
both sides of the plurality of stretchable elastic members (rubber threads) are adhered and fixed to contact points where peaks of the pleats (folds) and the plurality of the stretchable elastic members are in contact, the plurality of stretchable elastic members (rubber threads) being positioned in an orthogonal direction with respect to the peaks of the double-layer pleats (folds).

2. A method for manufacturing a stretchable composite sheet in which double-layer pleats (folds) are formed with stretchable elastic members adhered to and fixed between two non-woven fabric sheets, characterized in that:
upon intermittently coating an adhesive to stretchable elastic members which are being conveyed in an stretched state, coating of adhesive is applied to entire circumferential surfaces of the stretchable elastic members during the conveyance.

3. An apparatus for manufacturing a stretchable composite sheet in which double-layer pleats (folds) are formed with stretchable elastic members adhered and fixed between two non-woven fabric sheets, the apparatus being characterized in that:
said apparatus comprises an adhesive intermittent coating device provided with
slit grooves through which stretchable elastic members in a stretched state are conveyed to pass, and
a valve mechanism for intermittently supplying adhesives to the slit grooves;
each of said slit grooves is formed at a base portion thereof with an expanded section for applying coating of adhesives to each of the stretchable elastic members (rubber threads) passing through the slit groove, said expanded section being larger than a cross section of each of the stretchable elastic members (rubber threads), thus forming an adhesive coating area; and
said adhesive intermittent coating device supplies the adhesives from both sides of the adhesive coating area.
